(19) United States
(12) Patent Application Publication
(10) Pub. No.: US 2007/0196290 A1
(43) Pub. Date: Aug. 23, 2007

(54) Uv filters in powder form

(76) Inventors: Frank Pflucker, Darmstadt (DE); Jorn Beck, Seeheim-Jugenheim (DE); Hansjurgen Driller, Gross-Umstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/591,531

(22) PCT Filed: Feb. 8, 2005
(86) PCT No.: PCT/EP05/01244

§ 371 Date: Sep. 1, 2006

(30) Foreign Application Priority Data
Mar. 3, 2004 (DE) 10 2004 010 313.5

(51) Int. Cl. A61K 8/37; A61K 8/42

(52) U.S. Cl. 424/59; 977/926

(57) ABSTRACT

The present invention relates to UV filters in powder form, obtainable by spray-drying or freeze-drying a dispersion comprising UV filters, processes for the preparation of UV filters in powder form, and compositions comprising UV filters in powder form.

The present invention relates to UV filters in powder form, obtainable by spray-drying or freeze-drying a dispersion of UV filters, processes for the preparation of UV filters in powder form, and compositions comprising UV filters in powder form.

UV filters play a major role in a number of applications, but in particular in cosmetics. Increasing requirements are being made here of the skin tolerance and the applicational properties of the UV filters. In the case of organic UV filters, for example, penetration of the skin by the UV filter should be avoided in order to keep the exposure of the skin to organic substances as low as possible. In order to circumvent this, it has been proposed, for example, in EP 1 382 328, WO 00/09652, WO 00/72806 and WO 00/71084 to encapsulate and thus to immobilise organic UV filters. The UV filters encapsulated in this way are accessible as dispersions.

Dispersions prove to be disadvantageous in a number of applications. The actual UV filter cannot be added as the pure substance, but instead always only in combination with the additives, for example water, dispersion auxiliaries or preservatives. The avoidance of preservatives and water very particularly increases the quantity and flexibility of formulations. In particular in cosmetics, the presence of the additives proves to be problematical since cosmetic compositions frequently have a ratio of solid to liquid phase, for example in the form of a binder, which is precisely matched to one another.

In order, for example, to provide compact-powder mixtures with UV protection, some of the binder is usually substituted by liquid UV filters or by the UV filter dispersion and then added to the base solid of the powder. The flexibility of the binder is restricted here and, on addition of the binder to the powder, a change in the solid/binder mixing ratios occurs. However, this can result in disadvantageous effects with respect to the applicational properties, such as, for example, in colour changes or an impairment of the skin feel in the case of topical application, which is undesired by the users.

The object was therefore to provide UV filters, in particular encapsulated liquid UV filters, in a form which facilitates direct use of the UV filters and at the same time does not adversely affect the properties of the UV filters, in particular the stability thereof. The latter applies, in particular, to liquid UV filters in encapsulated form, whose encapsulation is ideally retained here in order to avoid the undesired liberation of the UV filters present in the capsules. In order to improve the applicational properties, the UV filters should be in solid form, this is particularly desired for encapsulated UV filters. Encapsulated liquid UV filters in solid form would thus be available for incorporation into cosmetic formulations. Surprisingly, it has been found that these requirements are met by the present invention.

The invention accordingly relates to UV filters in powder form, obtainable by spray-drying or freeze-drying a dispersion comprising UV filters. The present invention furthermore relates to processes for the preparation of UV filters in powder form, in which dispersions comprising UV filters are spray-dried or freeze-dried. The present invention furthermore likewise relates to compositions comprising UV filters in powder form.

The powder-form UV filters according to the invention have the advantage that they can be handled and processed better than dispersions. Thus, the powders according to the invention can readily be incorporated both into aqueous and also into non-aqueous phases, which is not possible with a dispersion. In addition, the possibility of specific modification of the surface, for example in the form of hydrophobicisation or hydrophilisation, in order further to improve the applicational properties is available in the case of UV filters in powder form. Since the powders according to the invention are dry, addition of preservatives is not necessary, which is essential in the case of dispersions in order to prevent nucleation of the dispersion. The achievement of freedom from nuclei by setting a low pH in the dispersion is also entirely usual, but proves to be problematical, in particular in cosmetics, since the pH of the end application plays a particular role there. This is avoided in the case of the UV filters according to the invention since the powder can be employed directly. A further advantage of the powders according to the invention is the considerable compaction compared with a dispersion, which, in particular, considerably reduces the transport costs. The removal of the dispersion medium, for example water or ethanol, considerably reduces the volume and weight of the UV filter to be transported, which has a considerable positive effect on the transport and storage costs. In addition, on use of UV filters in powder form, they can be added to compact-powder mixtures, with the binder simultaneously being optimised with respect to its skin feel, without an additional proportion of liquid UV filters having to be taken into account.

The invention relates to UV filters in powder form, obtainable by spray-drying or freeze-drying a dispersion comprising UV filters. The UV filter here may be an encapsulated and/or unencapsulated organic UV filter. Preference is given to particulate UV filters, with encapsulated UV filters, in particular encapsulated organic UV filters, preferably being employed, where the encapsulation is preferably inorganic. UV filters of this type were hitherto only available in the form of dispersions, which restricts the range of applications and makes the preparation of compositions more difficult since the dispersion medium and its properties always has to be taken into account in the preparation of the compositions. This disadvantage does not apply in the case of the powder-form UV filters according to the invention.

The size of the UV filters in particulate form is in the range from 10 nm to 100 μm, preferably in the range from 10 nm to 30 μm (d₅₀≦30 μm, measured by means of laser diffraction in water, for example using a Malvern Particle Sizer), in particular in the range from 0.1 μm to 20 μm.

Suitable unencapsulated organic UV filters are, for example, 4-amino-benzoic acid, 2-phenylbenzimidazole-5-sulfonic acid, 3-(4′-methylbenzylidene)-dl-camphor, 2,4,6-trianilino(p-carbo-2′-ethylhexyl-1′-oxy)-1,3,5-triazine or compounds from the group of the dibenzoylmethane derivatives or benzophenones, such as, for example, 2,4-dihydroxybenzophenone, 2-hydroxy-4-methoxybenzophenone.

Preference is given to the use of encapsulated UV filters, in particular encapsulated organic UV filters. In detail, the following advantages arise through the encapsulation:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin are repeatedly discussed in the literature. The encapsulation of the corresponding substances which is proposed here suppresses this effect.
In general, encapsulation of individual UV filters or other ingredients enables preparation problems caused by the interaction of individual composition constituents with one another, such as crystallisation processes, precipitation and agglomerate formation, to be avoided since the interaction is suppressed.

It may therefore be preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications EP 1 382 328, WO 00/09652, WO 00/72806 and WO 00/71084. Preference is again given here to capsules whose walls are built up from silica gel (silica; undefined silicon oxide hydroxide) or silicon dioxide. The production of corresponding capsules is known to the person skilled in the art, for example from the cited patent applications, whose contents expressly also belong to the subject-matter of the present application.

Suitable UV filters for the encapsulation are, for example, dibenzoylmethane derivatives.

The dibenzoylmethane derivatives which can be used in accordance with the invention may be selected, in particular, from the dibenzoylmethane derivatives of the following formula: 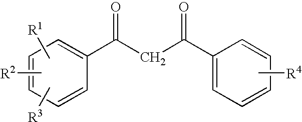
in which R¹, R², R³ and R⁴, which are identical or different from one another, denote hydrogen, a straight-chain or branched C₁₋₈-alkyl group or a straight-chain or branched C₁₋₈-alkoxy group. In accordance with the present invention, it is of course possible to use one dibenzoylmethane derivative or a plurality of dibenzoylmethane derivatives. Of the dibenzoylmethane derivatives to which the present invention more specifically relates, mention may be made, in particular, of:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-methoxy-tert-butyldibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane
and
2,6-dimethyl-4-tert-butyl-4′-methoxydibenzoylmethane,
   where this list is not restrictive.

Of the above-mentioned dibenzoylmethane derivatives, particular preference is given in accordance with the invention to 4,4′-methoxy-tert-butyldibenzoylmethane and especially 4,4′-methoxy-tert-butyldibenzoylmethane, which is commercially available under the trade name Eusolex® 9020 from Merck KGaA, this filter conforming to the following structural formula:

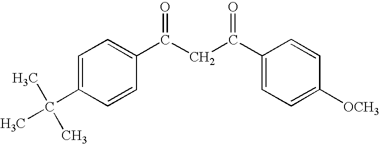

A further dibenzoylmethane derivative which is preferred in accordance with the invention is 4-isopropyldibenzoylmethane.

The capsules may of course also comprise one or more sunscreen filters which are effective in the UV-A region and/or UV-B region and/or IR and/or VIS region (absorbers). These filters can be selected, in particular, from cinnamic acid derivatives, salicylic acid derivatives, camphor derivatives, triazine derivatives, β,β-diphenyl acrylate derivatives, p-aminobenzoic acid derivatives and polymeric filters and silicone filters, which are described in the application WO 93/04665. Further examples of organic filters are indicated in patent application EP-A 0 487 404.

In principle, all UV filters are suitable for encapsulation. Particular preference is given to UV filters whose physiological acceptability has already been demonstrated. Both for UVA and UVB filters, there are many proven substances which are known from the specialist literature, for example benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL), methoxycinnamic acid esters, such as octyl methoxycinnamate (for example Eusolex® 2292), isopentyl 4-methoxycinnamate, for example as a mixture of the isomers (for example Neo Heliopan® E 1000), salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS), 4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25), phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid, and further substances, such as
2-ethylhexyl 2-cyano-3,3-diphenylacrylate (for example Eusolex® OCR),
3,3′-(1,4-phenylenedimethylene)bis-7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

Further suitable UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approx. 6% of methyl[2-[p-[2,2-bis(ethoxycarbonyl]vinyl]phenoxy]-1-methyleneethyl] and approx. 1.5% of methyl[3-[p-[2,2-bis(ethoxycarbonyl)vinyl)phenoxy)propenyl) and 0.1 to 0.4% of (methylhydrogen]silylene]] (n≈60) (CAS No. 207 574-74-1)
2,2′-methylenebis(6-(2H-benzotriazol-2-yl)-4-(1,1,3,3-tetramethylbutyl)phenol) (CAS No. 103 59745-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

Preferred capsules may also comprise compounds of the formula I 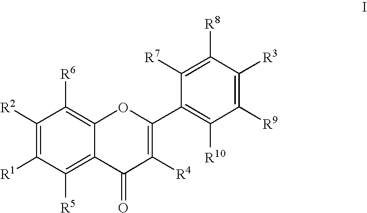
where R¹ and R² are selected from
   H
   and OR¹¹, where OR¹¹, independently of one another, stands for
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen, and/or
      C₃- to C₁₀-cycloalkoxy groups and/or C₃- to C₁₂-cycloalkenyloxy groups, where the rings may each also be bridged by —(CH₂)ₙ— groups, where n=1 to 3, and/or
      mono- and/or oligoglycosyl radicals,
with the proviso that at least one radical from R¹ and R² stands for OR¹¹,
and R³ stands for a radical OR¹¹ and
R⁴ to R¹ and R¹⁰ may be identical or different and, independently of one another, stand for
   H
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ— groups, where n=1 to 3, and
R⁸ and R⁹ may be identical or different and, independently of one another, stand for
   H
   OR¹¹
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ— groups, where n=1 to 3.

The flavonoids of the formula I to be employed in accordance with the invention include broad-band UV filters, other likewise preferred compounds of the formula I exhibit an absorption maximum in the boundary region between UV-B and UV-A radiation. As UV-A-II filters, they therefore advantageously supplement the absorption spectrum of commercially available UV-B and UV-A-I filters. Preferred capsules according to the invention comprise at least one compound of the formula I, where R³ stands for
OH or
straight-chain or branched C₁- to C₂₀-alkoxy groups, preferably methoxy, ethoxy or ethylhexyloxy, or
mono- and/or oligoglycosyl radicals, preferably glucosyl radicals, and
R¹ and/or R² preferably stand for
   OH or
   straight-chain or branched C₁- to C₂₀-alkoxy groups, preferably methoxy, ethoxy or ethylhexyloxy, or
   mono- and/or oligoglycosyl radicals, preferably glucosyl radicals.

These preferred compounds are distinguished by particularly intense UV absorption. It has been found that the intensity of the UV absorption is high, in particular, if R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy.

It is of course possible for the above-mentioned UV filters to be present alone in the capsules, but mixtures of a plurality of the UV filters mentioned may also be in the capsules. In addition, the UV filters in the capsules may also be combined with further substances, such as, for example, photostabilisers, cosmetic oils and/or antioxidants, in order to achieve an increase in the stability of the said UV filters. Examples and preferred compounds for the further substances mentioned above, in particular for the photostabilisers, are found in the remainder of this application under the general description of these substances. Examples of suitable cosmetic oils are mineral oils, mineral waxes, oils, such as triglycerides of capric or caprylic acid, furthermore natural oils, such as, for example, castor oil; fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids, silicone oils, such as, for example, dimethylpolysiloxanes, diethylpolysiloxanes, diphenylpolysiloxanes and mixed forms thereof.

The powder form is of particular interest for the encapsulated UV filters mentioned above since UV filters in capsule form are usually only available as a dispersion which has the above-mentioned disadvantages in the applications. Furthermore, the encapsulated UV filters are frequently in the form of ethanol-containing dispersions, which additionally restricts the area of application since ethanol is undesired as constituent in many applications, in particular in cosmetics. The powder-form UV filters according to the invention are preferably solvent-free or have a proportion of solvent which does not interfere in the respective applications and can thus be employed universally. For this reason, the powder-form UV filters according to the invention prove to be particularly advantageous.

The powders according to the invention may additionally be after-treated in a further embodiment of the present invention in order to modify the surface of the individual particles of the powder. Thus, the application of corresponding compounds enables hydrophobicisation or hydrophilisation of the particle surfaces to be achieved. Suitable for hydrophobic modification is, for example, coating with organic acids, such as, for example, stearic acid or lauric acid, with LCST polymers, organic fluoroalcohol phosphates or silicone or silane coating.

The silicones are, as is known, organosilicon polymers or oligomers having a straight-chain or cyclic, branched or crosslinked structure with various molecular weights which are obtained by polymerisation and/or polycondensation with suitably functionalised silanes and are essentially formed from recurring main units in which the silicon atoms are linked to one another via oxygen atoms (siloxane bond), where optionally substituted hydrocarbon groups are bonded directly to the silicon atoms via a carbon atom. The commonest hydrocarbon groups are alkyl groups and in particular methyl groups, fluoroalkyl groups, aryl groups and in particular phenyl groups, and alkenyl groups and in particular vinyl groups. Further types of group which can be bonded to the siloxane chain either directly or via a hydrocarbon group are, in particular, hydrogen, the halogens and in particular chlorine, bromine or fluorine, the thiols, alkoxy groups, polyoxyalkylene groups (or polyethers) and in particular polyoxyethylene and/or polyoxypropylene, hydroxyl groups or hydroxyalkyl groups, optionally substituted amino groups, amide groups, acyloxy groups or acyloxyalkyl groups, hydroxyalkylamino groups or aminoalkyl groups, quaternary ammonium groups, amphoteric groups or betaine groups, anionic groups, such as carboxylates, thioglycolates, sulfosuccinates, thiosulfates, phosphates and sulfates, where this list is of course in no way limiting (so-called ‘organo-modified’ silicones).

For the purposes of the present invention, the term ‘silicones’ is also intended to encompass and cover the silanes and in particular the alkylsilanes required for their preparation.

The silicones which are suitable for the present invention, which can be used for sheathing the UV protectants, are preferably selected from the alkylsilanes, the polydialkylsiloxanes and the polyalkylhydrogenosiloxanes. The silicones are more preferably selected from octyltrimethylsilane, the polydimethylsiloxanes and the polymethylhydrogenosiloxanes.

The present invention furthermore relates to processes for the preparation of UV filters in powder form, in which dispersions of UV filters are spray-dried or freeze-dried. Besides the spray-drying variants described, freeze-drying, it is also possible to use fluidised-bed drying (fluidised-bed granulation). Furthermore, all methods described in accordance with the prior art for the gentle drying of suspensions can be used. Spray-drying is preferably employed. The suitable types of UV filter have already been mentioned in the description of the powders according to the invention.

The processes according to the invention have the advantage that UV filters in powder form can be prepared in a gentle manner from corresponding dispersions. In particular in the case of the encapsulated organic UV filters, spray-drying proves to be a particularly suitable method for the preparation of corresponding powders. The capsules are sensitive to mechanical and strong thermal stress, which results in the encapsulation breaking open and in release of the substances included therein. This is undesired since direct contact of the UV filters with the skin is to be avoided precisely in the case of topical applications of UV filters. The structural retention of the encapsulation is thus of fundamental importance and represents a major challenge for these systems. Through the spray-drying technique preferably used in the present invention, this requirement has, surprisingly, also been met for these systems, so that UV filters of any type have thus successfully been prepared as powders, which represents a fundamental advantage, in particular in the case of encapsulated systems.

The respective UV filters are introduced into the spray drying in the form of dispersions. Basically all solvents are suitable here as dispersion media, such as, for example, water or organic solvents. Preference is given to aqueous dispersions since undesired solvent residues, which possibly make use of the powders in cosmetics more difficult or even prevent it, cannot remain in the powders here. In addition, there are no safety-relevant restrictions, such as, for example, with respect to inflammability or the risk of explosion, in the case of aqueous dispersions.

Suitable for spray-drying or freeze-drying are all process variants known to the person skilled in the art and all apparatuses suitable therefor, preferably spray-drying. The spray-drying process basically always includes four basic steps, namely droplet production, droplet/gas mixing, separation of the agglomerates and deposition of the fines. Process variants arise herefrom in the individual sub-steps. The droplet production can be carried out, for example, by centrifugal atomisation, one-component nozzle atomisation or two-component nozzle atomisation, in which product/gas transport can be carried out in co-current or countercurrent. In the present invention, preference is given to the use of centrifugal atomisation and two-component nozzle atomisation, with either co-current or countercurrent transport. Particular preference is given to the use of two-component nozzle atomisation in countercurrent mode or centrifugal atomisation with co-current transport. The product discharge can likewise be carried out by all process variants known to the person skilled in the art, preferably two-point deposition, i.e., for example, a dryer cone and a cyclone are used.

The entry temperature for aqueous dispersions can be in the range from 110° C. to 200° C., preferably in the range from 130° C. to 160° C., and in particular in the range from 140° C. to 150° C. The exit temperature for the powder can be in the range from 40 to 90° C., preferably in the range from 50 to 80° C. If solvent-containing dispersions are employed, the entry and exit temperatures can be selected to be correspondingly lower and thus matched individually to the solvent. The ratio of spray-material throughput to quantity of drying air can be in the range from 0.5 kg/h:200 m³/h to 5 kg/h:50 m³/h, the ratio is preferably between 0.8 kg/h:70 m³/h to 1.5 kg/h:75 m³/h.

In order to modify the product properties and in order to modify the spray properties, corresponding additives can be introduced into the processes according to the invention before or during the spray-drying or freeze-drying, for example as addition to the dispersions employed or directly as additive during the process. A very wide variety of auxiliaries known to the person skilled in the art, such as sugar alcohols, polyols, corn starch, cyclodextrins, emulsifiers, surfactants, cellulose derivatives, xanthan gum, PVP, can be used here. The amount of the additives added can be 0.01-20% by weight, preferably 0.1-10% by weight.

In a further embodiment, the UV filters in powder form are after-treated. The after-treatment can be carried out in all manners known to the person skilled in the art, such as, for example, by polymer precipitation methods in aqueous or organic media, by solvolysis methods, by dispersion methods or by simple mixing.

The powder-form UV filters according to the invention are basically suitable for use in any form of composition, such as, for example, cosmetic compositions, but also in compositions which can be employed in the industrial sector, such as, for example, paints or coatings. The powder-form UV filters according to the invention are preferably employed in compositions in cosmetics, in particular in decorative cosmetics. The powder-form UV filters according to the invention allow the preparation of compositions having light-protection properties for a multiplicity of application variants and media which can only be prepared with difficulty using the dispersions known from the prior art since the dispersion medium considerably changes the composition of the compositions. Thus, UV filters which are suitable for use in compact powders can be prepared by the processes according to the invention without changing or limiting the skin feel or the composition possibilities. The proportion of the UV filters in powder form in these compositions can be 0.1 to 30% by weight and preferably 0.5 to 15% by weight, based on the composition. The powder-form UV filters according to the invention can be incorporated either into the aqueous or oily phase of a composition.

Preferred compositions having light-protection properties additionally comprise at least one further organic UV filter here, preferably a dibenzoylmethane derivative. The dibenzoylmethane derivatives used for the purposes of the present invention are products which are already well known per se and are described, in particular, in the specifications FR-A-2 326 405, FR-A-2 440 933 and EP-A-0 114 607.

The dibenzoylmethane derivatives which can be used in accordance with the invention can be selected, in particular, from the dibenzoylmethane derivatives of the following formula: 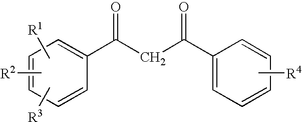
in which R¹, R², R³ and R⁴, which are identical or different from one another, denote hydrogen, a straight-chain or branched C₁₋₈-alkyl group or a straight-chain or branched C₁₋₈alkoxy group. In accordance with the present invention, it is of course possible to use one dibenzoylmethane derivative or a plurality of dibenzoylmethane derivatives. Of the dibenzoylmethane derivatives to which the present invention specifically relates, mention may be made, in particular, of:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-methoxy-tert-butyldibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane
and
2,6-dimethyl-4-tert-butyl-4′-methoxydibenzoylmethane,
   this list being non-restrictive.

Of the above-mentioned dibenzoylmethane derivatives, particular preference is given in accordance with the invention to 4,4′-methoxy-tert-butyldibenzoylmethane and especially 4,4′-methoxy-tert-butyldibenzoylmethane, which is commercially available under the trade name Eusolex® 9020 from Merck KGaA, this filter conforming to the following structural formula:

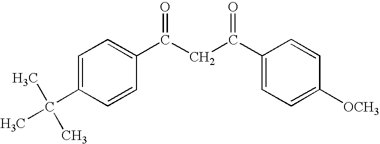

A further dibenzoylmethane derivative which is preferred in accordance with the invention is 4-isopropyldibenzoylmethane.

Further preferred compositions having light-protection properties comprise at least one benzophenone or benzophenone derivatives, such as, particularly preferably, 2-hydroxy-4-methoxybenzophenone (for example Eusolex® 4360) or 2-hydroxy-4-methoxybenzophenone-5-sulfonic acid and the sodium salt thereof (for example Uvinul® MS-40).

The dibenzoylmethane derivative(s) or the benzophenone derivative(s) may be present in the compositions according to the invention in proportions which are generally in the range from 0.1 to 10% by weight and preferably in proportions which are in the range from 0.3 to 5% by weight, where these proportions are based on the total weight of the composition.

Owing to the above-mentioned advantages, the present invention furthermore also relates to the use of a powder-form UV filter for preventing destabilisation of other UV filters, in particular dibenzoylmethane and dibenzoylmethane derivatives or benzophenone and benzophenone derivatives.

It may furthermore be preferred in accordance with the invention for the compositions to comprise further inorganic UV filters. Preference is given here both to those from the group of the titanium dioxides, such as, for example, coated titanium dioxide (for example Eusolex® T-2000, Eusolex® T-AQUA), zinc oxides (for example Sachtotec®), iron oxides or also cerium oxides. These inorganic UV filters are generally incorporated into cosmetic compositions in an amount of 0.5 to 20 percent by weight, preferably 2-10%. In particular, it may be preferred here for a powder-form UV filter according to the invention to be incorporated into one phase in emulsions and for a further inorganic UV filter to be incorporated into the other phase.

In accordance with the invention, the above-mentioned UV filters can also be provided with a surface treatment which augments the hydrophilic or hydrophobic properties. Examples of surface treatments of this type have already been mentioned.

The UV protectants can be present in the compositions according to the invention in proportions which are generally in the range from 0.1 to 50% by weight and preferably in proportions which are in the range from 0.5 to 20% by weight, where these proportions are based on the total weight of the composition.

In a further, likewise preferred embodiment of the present invention, the composition according to the invention comprises at least one self-tanning agent.

Advantageous self-tanning agents which can be employed are, inter alia:

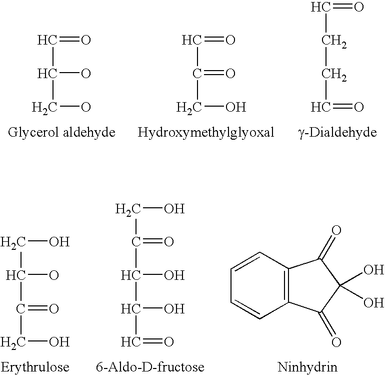

Mention should also be made of 5-hydroxy-1,4-naphthoquinone (juglone), which is extracted from the shells of fresh walnuts 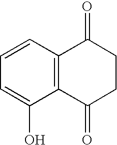
5-Hydroxy-1,4-naphthoquinone (juglone)
and 2-hydroxy-1,4-naphthoquinone (lawsone), which occurs in henna leaves. 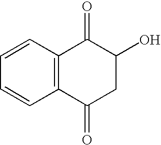
2-Hydroxy-1,4-naphthoquinone (lawsone)

Very particular preference is given to 1,3-dihydroxyacetone (DHA), a tri-functional sugar which occurs in the human body, and derivatives thereof. 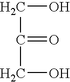
1,3-Dihydroxyacetone (DHA)

The present invention furthermore relates to the use of a powder-form UV filter according to the invention in the stabilisation of self-tanning agents, in particular dihydroxyacetone or dihydroxyacetone derivatives.

Furthermore, the compositions according to the invention having light-protection properties may also comprise dyes and coloured pigments. The dyes and coloured pigments can be selected from the corresponding positive list in the German Cosmetics Regulation or the EC list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-Dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′-sulfodiethylamido-1′-phenylazo)-3- | 12490 | Red |
| hydroxy-5″-chloro-2″,4″-dimethoxy-2-naphthanilide | | |
| Disperse Yellow 16 | 12700 | Yellow |
| 1-(4-Sulfo-1-phenylazo)-4-aminobenzene-5-sulfonic acid | 13015 | Yellow |
| 2,4-Dihydroxyazobenzene-4′-sulfonic acid | 14270 | Orange |
| 2-(2,4-Dimethylphenylazo-5-sulfonyl)-1-hydroxynaphthalene- | 14700 | Red |
| 4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2- | 15525 | Red |
| hydroxynaphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic | 15850 | Red |
| acid | | |
| 1-(2-Sulfo-4-methyl-5-chloro-1-phenylazo)-2-hydroxynaphthalene- | 15865 | Red |
| 3-carboxylic acid | | |
| 1-(2-Sulfo-1-naphthylazo)-2-hydroxynaphthalene-3- | 15880 | Red |
| carboxylic acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic | 16290 | Red |
| acid | | |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo)-1,3-dihydroxy- | 20170 | Orange |
| benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′-naphthylazo]-1- | 27755 | Black |
| hydroxy-7-aminonaphthalene-3,6-disulfonic acid | | |
| 4-[4″-Sulfo-1″-phenylazo)-7′-sulfo-1′-naphthylazo]-1- | 28440 | Black |
| hydroxy-8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″- | 42051 | Blue |
| bis(diethylamino)triphenylcarbinol | | |
| 4-[(4-N-Ethyl-p-sulfobenzylamino)-phenyl-(4-hydroxy-2- | 42053 | Green |
| sulfophenyl)(methylene)-1-(N-ethyl-N-p-sulfobenzyl)-2,5- | | |
| cyclohexadienimine] | | |
| Acid Blue 7 | 42080 | Blue |
| (N-Ethyl-p-sulfobenzylamino)phenyl-(2-sulfophenyl)- | 42090 | Blue |
| methylene-(N-ethyl-N-p-sulfobenzyl)-Δ^{2,5}-cyclohexadienimine | | |
| Acid Green 9 | 42100 | Green |
| Diethyldisulfobenzyldi-4-amino-2-chlorodi-2-methyl- | 42170 | Green |
| fuchsonimmonium | | |
| Basic Violet 14 | 42510 | Violet |
| Basic Violet 2 | 42520 | Violet |
| 2′-Methyl-4′-(N-ethyl-N-m-sulfobenzyl)amino-4″-(N- | 42735 | Blue |
| diethyl)amino-2-methyl-N-ethyl-N-m-sulfobenzyl- | | |
| fuchsonimmonium | | |
| 4′-(N-Dimethyl)amino-4″-(N-phenyl)aminonaphtho-N-di- | 44045 | Blue |
| methylfuchsonimmonium | | |
| 2-Hydroxy-3,6-disulfo-4,4′-bisdimethylaminonaphtho- | 44090 | Green |
| fuchsonimmonium | | |
| Acid Red 52 | 45100 | Red |
| 3-(2′-Methylphenylamino)-6-(2′-methyl-4′-sulfophenyl- | 45190 | Violet |
| amino)-9-(2″-carboxyphenyl)xanthenium salt | | |
| Acid Red 50 | 45220 | Red |
| Phenyl-2-oxyfluorone-2-carboxylic acid | 45350 | Yellow |
| 4,5-Dibromofluoroescein | 45370 | Orange |
| 2,4,5,7-Tetrabromofluoroescein | 45380 | Red |
| Solvent Dye | 45396 | Orange |
| Acid Red 98 | 45405 | Red |
| 3′,4′,5′,6′-Tetrachloro-2,4,5,7-tetrabromofluoroescein | 45410 | Red |
| 4,5-Diiodofluoroescein | 45425 | Red |
| 2,4,5,7-Tetraiodofluoroescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-Dioxyanthraquinone, calcium-aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-Di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-Dihydro-1,2,1′,2′-anthraquinonazine | 69800 | Blue |
| Vat Blue 6; Pigment Blue 64 | 69825 | Blue |
| Vat Orange 7 | 71105 | Orange |
| Indigo | 73000 | Blue |
| Indigodisulfonic acid | 73015 | Blue |
| 4,4′-Dimethyl-6,6′-dichlorothioindigo | 73360 | Red |
| 5,5′Dichloro-7,7′-dimethylthioindigo | 73385 | Violet |
| Quinacridone Violet 19 | 73900 | Violet |
| Pigment Red 122 | 73915 | Red |
| Pigment Blue 16 | 74100 | Blue |
| Phthalocyanine | 74160 | Blue |
| Direct Blue 86 | 74180 | Blue |
| chlorinated phthalocyanine | 74260 | Green |
| Natural Yellow 6, 19; Natural Red 1 | 75100 | Yellow |
| Bixin, Nor-Bixin | 75120 | Orange |
| Lycopene | 75125 | Yellow |
| trans-alpha-, -beta- or -gamma-Carotene | 75130 | Orange |
| Keto and/or hydroxyl derivatives of carotene | 75135 | Yellow |
| Guanine or pearlescent agent | 75170 | White |
| 1,7-Bis(4-hydroxy-3-methoxyphenyl)-1,6-heptadiene-3,5- | 75300 | Yellow |
| dione | | |
| Complex salt (Na, Al, Ca) of carminic acid | 75470 | Red |
| chlorophyll a and b; copper compounds of chlorophylls | 75810 | Green |
| and chlorophyllines | | |
| Aluminium | 77000 | White |
| Aluminium hydroxide | 77002 | White |
| Water-containing aluminium silicates | 77004 | White |
| Ultramarine | 77007 | Blue |
| Pigment Red 101 and 102 | 77015 | Red |
| Barium sulfate | 77120 | White |
| Bismuth oxychloride and mixtures thereof with mica | 77163 | White |
| Calcium carbonate | 77220 | White |
| Calcium sulfate | 77231 | White |
| Carbon | 77266 | Black |
| Pigment Black 9 | 77267 | Black |
| Carbo medicinalis vegetabilis | 77268: 1 | Black |
| Chromium oxide | 77288 | Green |
| Chromium oxide, water-containing | 77278 | Green |
| Pigment Blue 28, Pigment Green 14 | 77346 | Green |
| Pigment Metal 2 | 77400 | Brown |
| Gold | 77480 | Brown |
| Iron oxides and hydroxides | 77489 | Orange |
| Iron oxide | 77491 | Red |
| Iron oxide hydrate | 77492 | Yellow |
| Iron oxide | 77499 | Black |
| Mixtures of iron(II) and iron(III) hexacyanoferrate | 77510 | Blue |
| Pigment White 18 | 77713 | White |
| Manganese ammonium diphosphate | 77742 | Violet |
| Manganese phosphate; Mn₃(PO₄)₂•7 H₂O | 77745 | Red |
| Silver | 77820 | White |
| Titanium dioxide and mixtures thereof with mica | 77891 | White |
| Zinc oxide | 77947 | White |
| 6,7-Dimethyl-9-(1′-D-ribityl)isoalloxazine, lactoflavin | | Yellow |
| Sugar dye | | Brown |
| Capsanthin, capsorubin | | Orange |
| Betanin | | Red |
| Benzopyrylium salts, anthocyans | | Red |
| Aluminium, zinc, magnesium and calcium stearate | | White |
| bromothymol Blue | | Blue |

It may furthermore be favourable to select, as dye, one or more substances from the following group:

2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluoroescein, the aluminium and zirconium salts of 2,4,5,7-tetrabromofluoroescein, 3′,4′,5′,6′-tetrachloro-2,4,5,7-tetrabromofluoroescein and the aluminium salt thereof, the aluminium salt of 2,4,5,7-tetraiodofluoroescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising effect pigments. Particular preference is given to the types of effect pigment listed below:
1. Natural effect pigments, such as, for example,
   a) “pearl essence” (guanine/hypoxanthine mixed crystals from fish scales) and
   b) “mother-of-pearl” (ground mussel shells)
2. Monocrystalline effect pigments, such as, for example, bismuth oxychloride (BiOCl)
3. Layered substrate pigments: for example mica/metal oxide

The basis for effect pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following effect pigment types based on mica/metal oxide:

| | Coating/layer | |
|---|---|---|
| Group | thickness | Colour |
| Silver-white effect pigments | TiO₂: 40-60 nm | silver |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck KGaA under the trade names Timiron®, Colorona® or Dichrona®.

The list of the said effect pigments is of course not intended to be limiting. Effect pigments which are advantageous for the purposes of the present invention can be obtained by numerous routes known per se. In addition, for example, other substrates apart from mica can also be coated with further metal oxides, such as, for example, silica and the like. For example, TiO₂- and Fe₂O₃-coated SiO₂ particles (“Ronasphere” grades), which are marketed by Merck KGaA and are particularly suitable for the optical reduction of fine wrinkles, are advantageous.

It may additionally be advantageous to completely omit a substrate such as mica. Particular preference is given to effect pigments prepared using SiO₂. Such pigments, which may additionally also have goniochromatic effects, are available, for example, from Merck KGaA under the trade name Colorstream®.

It may also be advantageous to employ Engelhard pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green, blue). The glitter particles here are in the form of mixtures with various auxiliaries and dyes (such as, for example, the dyes with the Colour Index (CI) numbers 19140, 77007, 77289, 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably from 0.5 to 15% by weight, in particular from 1.0 to 10% by weight, in each case based on the total weight of the compositions.

The compositions according to the invention having light-protection properties may of course comprise one or more additional hydrophilic or lipophilic sunscreen filters which are effective in the UV-A region and/or UV-B region and/or IR and/or VIS region (absorbers). These additional filters can be selected, in particular, from cinnamic acid derivatives, salicylic acid derivatives, camphor derivatives, triazine derivatives, β,β-diphenyl acrylate derivatives, p-aminobenzoic acid derivatives and polymeric filters and silicone filters, which are described in the application WO 93/04665. Further examples of organic filters are indicated in patent application EP-A 0 487 404.

In principle, all UV filters are suitable for combination with the UV protectants present in the powders according to the invention. Particular preference is given to UV filters whose physiological acceptability has already been demonstrated. Both for UVA and UVB filters, there are many proven substances which are known from the specialist literature, for example benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL), methoxycinnamic acid esters, such as octyl methoxycinnamate (for example Eusolex® 2292), isopentyl 4-methoxycinnamate, for example as a mixture of the isomers (for example Neo Heliopan® E 1000), salicylate derivatives, such as 2-ethylhexyl salicylate (for example Eusolex® OS), 4-isopropylbenzyl salicylate (for example Megasol® or 3,3,5-trimethylcyclohexyl salicylate (for example Eusolex® HMS), 4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25), phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid; and further substances, such as
2-ethylhexyl 2-cyano-3,3-diphenylacrylate (for example Eusolex® OCR),
3,3′-(1,4-phenylenedimethylene)bis-7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters. In particular, organic particulate UV filters, as described, for example, in patent application WO 99/66896, may also advantageously be combined with the powders according to the invention.

These organic UV filters are generally incorporated into cosmetic formulations in an amount of 0.5 to 20 percent by weight, preferably 1-10% by weight.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approx. 6% of methyl[2-[p-[2,2-bis(ethoxycarbonyl]vinyl]phenoxy]-1-methyleneethyl] and approx. 1.5% of methyl[3-[p-[2,2-bis(ethoxycarbonyl)vinyl)phenoxy)propenyl) and 0.1 to 0.4% of (methylhydrogen]silylene]] (n≈60) (CAS No. 207 574-74-1)
2,2′-methylenebis(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

Preferred compositions may also comprise compounds of the formula I 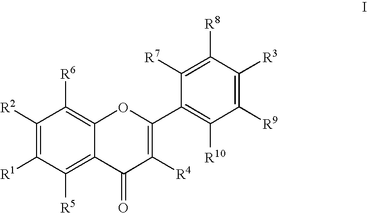
where R¹ and R² are selected from
   H
   and OR¹¹, where OR¹¹, independently of one another, stands for
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen, and/or
      C₃- to C₁₀-cycloalkoxy groups and/or C₃- to C₁₂-cycloalkenyloxy groups, where the rings may each also be bridged by —(CH₂)ₙ— groups, where n=1 to 3, and/or
      mono- and/or oligoglycosyl radicals,
with the proviso that at least one radical from R¹ and R² stands for OR¹¹,
and R³ stands for a radical OR¹¹ and
R⁴ to R⁷ and R¹⁰ may be identical or different and, independently of one another, stand for
   H
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₋₂-cycloalkenyl groups, where the rings may each also be bridged by —(CH₂)ₙ— groups, where n=1 to 3, and
R⁸ and R⁹ may be identical or different and, independently of one another, stand for
   H
   OR¹¹
   straight-chain or branched C₁- to C₂₀-alkyl groups,
   straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₋₂-cycloalkenyl groups, where the rings may each also be bridged by —(CH₂)ₙ— groups, where n=1 to 3.

Advantages of the compositions according to the invention are, in particular, the UV light-filtering action and the good toleration by the skin. In addition, the compounds of the formula I described here are colourless or only weakly coloured and thus, in contrast to many known naturally occurring flavonoids, do not result in discoloration of the compositions.

The flavonoids of the formula I to be employed in accordance with the invention include broad-band UV filters, other likewise preferred compounds of the formula I exhibit an absorption maximum in the boundary region between UV-B and UV-A radiation. As UV-A-II filters, they therefore advantageously supplement the absorption spectrum of commercially available UV-B and UV-A-1 filters. Preferred compositions according to the invention having light-protection properties comprise at least one compound of the formula I, where R³ stands for
OH or
straight-chain or branched C₁- to C₂₀-alkoxy groups, preferably methoxy, ethoxy or ethylhexyloxy, or
mono- and/or oligoglycosyl radicals, preferably glucosyl radicals, and
R¹ and/or R² preferably stand for
   OH or
   straight-chain or branched C₁- to C₂₀-alkoxy groups, preferably methoxy, ethoxy or ethylhexyloxy, or
   mono- and/or oligoglycosyl radicals, preferably glucosyl radicals.

These preferred compounds are distinguished by particularly intense UV absorption.

In addition, preferred compounds of this type have advantages on incorporation into the compositions:
mono- and/or oligoglycosyl radicals improve the water solubility of the compounds to be employed in accordance with the invention;
straight-chain or branched C₁- to C₂₀-alkoxy groups, in particular the long-chain alkoxy functions, such as ethylhexyloxy groups, increase the oil solubility of the compounds;
   i.e. the hydrophilicity or lipophilicity of the compounds of the formula I can be controlled via a suitable choice of the substituents. Preferred mono- or oligosaccharide radicals here are hexosyl radicals, in particular ramnosyl radicals and glucosyl radicals. However, other hexosyl radicals, for example allosyl, altrosyl, galactosyl, gulosyl, idosyl, mannosyl and talosyl, may also, if desired, advantageously be used. It may also be advantageous to use pentosyl radicals. The glycosyl radicals can be bonded to the parent structure α- or β-glycosidically. A preferred disaccharide is, for example, 6-O-(6-deoxy-α-L-mannopyranosyl)-β-D-glucopyranoside.

It has been found that the intensity of the UV absorption is particularly high if R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy.

Particular preference is therefore given in accordance with the invention to compositions having light-protection properties comprising at least one compound of the formula I which is characterised in that R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy. It is particularly preferred here if R⁸ and R⁹ stand for H.

The compounds of the formula I are typically employed in accordance with the invention in amounts of 0.01 to 20% by weight, preferably in amounts of 0.5% by weight to 10% by weight and particularly preferably in amounts of 1 to 8% by weight. The person skilled in the art is presented with absolutely no difficulties at all in correspondingly selecting the amounts depending on the intended light protection factor of the composition.

Combination of one or more nanoparticulate UV protectants with further UV filters in the powders according to the invention enables the protective action against harmful effects of UV radiation to be optimised. Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor.

All said UV filters, including the compounds of the formula I, can likewise also be employed in encapsulated form. In particular, it is advantageous to employ organic UV filters in encapsulated form. Examples of encapsulation have already been mentioned above under the description of the powder-form UV filters according to the invention. In addition, these capsules may also be after-treated, i.e. the surface of the particles is hydrophobicised or hydrophilised. Examples of after-treatments of this type are likewise already known.

If the compositions according to the invention comprise compounds of the formula I containing free hydroxyl groups, they additionally, besides the properties described, exhibit an action as antioxidant and/or free-radical scavenger. Preference is therefore also given to compositions having light-protection properties comprising at least one compound of the formula I which is characterised in that at least one of the radicals R¹ to R³ stands for OH, preferably with at least one of the radicals R¹ or R² standing for OH.

In order that the compounds of the formula I are able to develop their positive action as free-radical scavengers particularly well on the skin, it may be preferred to allow the compounds of the formula I to penetrate into deeper skin layers. Several possibilities are available for this purpose. Firstly, the compounds of the formula I can have an adequate lipophilicity in order to be able to penetrate through the outer skin layer into epidermal layers. As a further possibility, corresponding transport agents, for example liposomes, which enable transport of the compounds of the formula I through the outer skin layers may also be provided in the composition. Finally, systemic transport of the compounds of the formula I is also conceivable. The composition is then designed, for example, in such a way that it is suitable for oral administration.

In general, the substances of the formula I act as free-radical scavengers. Free radicals of this type are not generated only by sunlight, but instead are formed under various conditions. Examples are anoxia, which blocks the flow of electrons upstream of the cytochrome oxidases and causes the formation of superoxide free-radical anions; inflammation associated, inter alia, with the formation of superoxide anions by the membrane NADPH oxidase of the leucocytes, but also associated with the formation (through disproportionation in the presence of iron(II) ions) of the hydroxyl free radicals and other reactive species which are normally involved in the phenomenon of phagocytosis; and lipid autoxidation, which is generally initiated by a hydroxyl free radical and produces lipidic alkoxy free radicals and hydroperoxides.

It is assumed that preferred compounds of the formula I also act as enzyme inhibitors. They are thought to inhibit histidine decarboxylase, protein kinases, elastase, aldose reductase and hyaluronidase, and therefore enable the intactness of the basic substance of vascular sheaths to be maintained. Furthermore, they are thought to inhibit catechol O-methyl transferase non-specifically, causing the amount of available catecholamines and thus the vascular strength to be increased. Furthermore, they inhibit AMP phosphodiesterase, giving the substances potential for inhibiting thrombocyte aggregation.

Owing to these properties, the compositions according to the invention are, in general, suitable for immune protection and for the protection of DNA and RNA. In particular, the compositions are suitable for the protection of DNA and RNA against oxidative attack, against free radicals and against damage due to radiation, in particular UV radiation. A further advantage of the compositions according to the invention is cell protection, in particular protection of Langerhans cells against damage due to the influences mentioned above. The present invention also expressly relates to all these uses and to the use of the compounds of the formula I for the preparation of compositions which can be employed correspondingly.

In particular, preferred compositions according to the invention are also suitable for the treatment of skin diseases associated with a defect in keratinisation which affects differentiation and cell proliferation, in particular for the treatment of acne vulgaris, acne comedonica, polymorphic acne, acne rosaceae, nodular acne, acne conglobata, age-induced acne, acne which arises as a side effect, such as acne solaris, medicament-induced acne or acne professionalis, for the treatment of other defects in keratinisation, in particular ichthyosis, ichthyosiform states, Darier's disease, keratosis palmoplantaris, leukoplakia, leukoplakiform states, herpes of the skin and mucous membrane (buccal) (lichen), for the treatment of other skin diseases associated with a defect in keratinisation and which have an inflammatory and/or immunoallergic component and in particular all forms of psoriasis which affect the skin, mucous membranes and fingers and toenails, and psoriatic rheumatism and skin atopy, such as eczema or respiratory atopy, or hypertrophy of the gums, it furthermore being possible for the compounds to be used for some inflammation which is not associated with a defect in keratinisation, for the treatment of all benign or malignant excrescence of the dermis or epidermis, which may be of viral origin, such as verruca vulgaris, verruca plana, epidermodysplasia verruciformis, oral papillomatosis, papillomatosis florida, and excrescence which may be caused by UV radiation, in particular epithelioma baso-cellulare and epithelioma spinocellulare, for the treatment of other skin diseases, such as dermatitis bullosa and diseases affecting the collagen, for the treatment of certain eye diseases, in particular corneal diseases, for overcoming or combating light-induced skin ageing associated with ageing, for reducing pigmentation and keratosis actinica and for the treatment of all diseases associated with normal ageing or light-induced ageing, for the prevention or healing of wounds/scars of atrophy of the epidermis and/or dermis caused by locally or systemically applied corticosteroids and all other types of skin atrophy, for the prevention or treatment of defects in wound healing, for the prevention or elimination of stretch marks caused by pregnancy or for the promotion of wound healing, for combating defects in sebum production, such as hyperseborrhoea in acne or simple seborrhoea, for combating or preventing cancer-like states or pre-carcinogenic states, in particular promyelocytic leukaemia, for the treatment of inflammatory diseases, such as arthritis, for the treatment of all virus-induced diseases of the skin or other areas of the body, for the prevention or treatment of alopecia, for the treatment of skin diseases or diseases of other areas of the body with an immunological component, for the treatment of cardiovascular diseases, such as arteriosclerosis or hypertension, and of non-insulin-dependent diabetes, and for the treatment of skin problems caused by UV radiation.

The protective action of compositions according to the invention against oxidative stress or against the effect of free radicals can thus be further improved if the compositions comprise one or more antioxidants, where the person skilled in the art is presented with absolutely no difficulties at all in selecting suitable antioxidants which act quickly or in a delayed manner.

In a preferred embodiment of the present invention, the composition is therefore a composition for the protection of body cells against oxidative stress, in particular for reducing skin ageing, characterised in that it comprises one or more antioxidants in addition to the one or more compounds of the formula I.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such compositions with compounds of the formula I in ratios in the range from 1000:1 to 1:1000, preferably in amounts of 100:1 to 1:100.

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. Vitamins are usually employed here with compounds of the formula I in ratios in the range from 1000:1 to 1:1000, preferably in amounts of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I.M.C.M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A.E.M.F. Soffers, I.M.C.M. Rietjens; Free Radical Biology&Medicine 2001, 31(7), 869-881, are investigating the pH dependence of the antioxidant action of hydroxyflavones. Quercetin exhibits the greatest activity amongst the structures investigated over the entire pH range.

Suitable antioxidants are furthermore compounds of the formula II 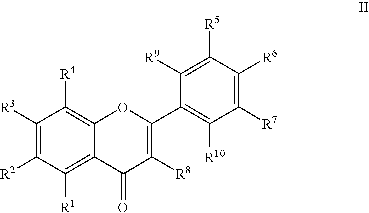
where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,
where all OR¹¹, independently of one another, stand for
OH
straight-chain or branched C₁- to C₂₀-alkoxy groups,
straight-chain or branched C₃- to C₂₀-alkenyloxy groups,
straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen, and/or
C₃- to C₁₀-cycloalkoxy groups and/or C₃- to C₁₂-cycloalkenyloxy groups, where the rings may each also be bridged by —(CH₂)ₙ— groups, where n=1 to 3, and/or
mono- and/or oligoglycosyl radicals,
   with the proviso that at least 4 radicals from R¹ to R⁷ stand for OH and that at least 2 pairs of adjacent —OH groups are present in the molecule,
or R², R⁵ and R⁶ stand for OH and the radicals R¹, R³, R⁴ and R⁷⁻¹⁰ stand for H,
   as described in the earlier German patent application DE 10244282.7.

Advantages of the compositions according to the invention comprising at least one antioxidant, besides the above-mentioned advantages, are, in particular, the antioxidant action and the good tolerance by the skin. In addition, preferred compounds of those described here are colourless or only weakly coloured and thus do not result in discoloration of the compositions, or only do so to a small extent. Particularly advantageous is the particular action profile of the compounds of the formula II, which is evident in the DPPH assay from a high capacity for scavenging free radicals (EC₅₀), a delayed action (T_{EC50}>120 min) and thus morate to high anti-free-radical efficiency (AE). In addition, the compounds of the formula II combine in the molecule antioxidative properties with UV absorption in the UV-A and/or -B region. Preference is therefore also given to compositions comprising at least one compound of the formula II which is characterised in that at least two adjacent radicals of the radicals R¹ to R⁴ stand for OH and at least two adjacent radicals of the radicals R⁵ to R⁷ stand for OH. Particularly preferred compositions comprise at least one compound of the formula II which is characterised in that at least three adjacent radicals of the radicals R¹ to R⁴ stand for OH, where the radicals R¹ to R³ preferably stand for OH.

In accordance with the invention, flavone derivatives are taken to mean flavonoids and coumaranones. In accordance with the invention, flavonoids are taken to mean the glycosides of flavonones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, this term is also taken to mean the aglycones, i.e. the sugar-free constituents, and the derivatives of the flavonoids and aglycones. For the purposes of the present invention, the term flavonoid is furthermore also taken to mean anthocyanidine (cyanidine). For the purposes of the present invention, the term coumaranones is also taken to mean derivatives thereof.

Preferred flavonoids are derived from flavonones, flavones, 3-hydroxyflavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin), α-glycosylrutin, tiliroside and the sulfates and phosphates thereof. Of the flavonoids, particular preference is given, as active substances according to the invention, to rutin, tiliroside, α-glycosylrutin and troxerutin.

Of the coumaranones, preference is given to 4,6,3′,4′-tetrahydroxybenzylcoumaranone-3.

The term chromone derivatives is preferably taken to mean certain chromen-2-one derivatives which are suitable as active ingredients for the preventative treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula III 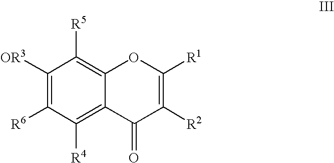
where
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups,
   where the rings may each also be bridged by —(CH₂)ₙ— groups, where n=1 to 3,
   R³ stands for H or straight-chain or branched C₁- to C₂₀-alkyl groups,
   R⁴ stands for H or OR⁸,
   R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen and
   R⁷ stands for H, straight-chain or branched C₁- to C₂₀-alkyl groups, a polyhydroxyl compound, such as preferably an ascorbic acid radical or glycosidic radicals, and
   R⁸ stands for H or straight-chain or branched C₁- to C₂₀-alkyl groups, where at least 2 of the substituents R¹, R², R⁴-R⁵ are not H or at least one substituent from R¹ and R² stands for —C(═O)—R⁷ or —C(═O)—OR⁷.

The proportion of one or more compounds selected from flavonoids, chromone derivatives and coumaranones in the composition according to the invention is preferably from 0.001 to 5% by weight, particularly preferably from 0.01 to 2% by weight, based on the composition as a whole.

The compositions having light-protection properties according to the invention may in addition comprise further conventional skin-protecting or skincare active ingredients. These can in principle be any active ingredients known to the person skilled in the art.

Particularly preferred active ingredients are pyrimidinecarboxylic acids and/or aryl oximes.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutical active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula IV 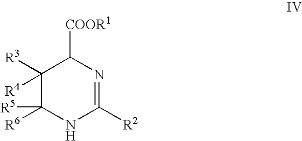
in which R¹ is a radical H or C1-8-alkyl, R² is a radical H or C1-4-alkyl, and R³, R⁴, R⁵ and R⁶ are each, independently of one another, a radical from the group H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight.

Of the aryl oximes, preference is given to the use of 2-hydroxy-5-methyllaurophenone oxime, which is also known as HMLO , LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which comprise aryl oximes, preferably 2-hydroxy-5-methyllaurophenone oxime, exhibit surprising antiinflammatory suitability. The compositions here preferably comprise 0.01 to 10% by weight of the aryl oxime, it being particularly preferred for the composition to comprise 0.05 to 5% by weight of aryl oxime.

All compounds or components described here that can be used in the compositions are either known and commercially available or can be synthesised by known processes.

Besides the compounds described here, the compositions according to the invention may also comprise at least one photostabiliser, preferably conforming to the formula V 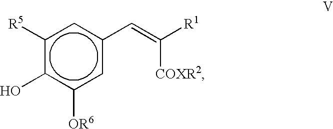
where
R¹ is selected from —C(O)CH₃, —CO₂R³, —C(O)NH₂ and —C(O)N(R⁴)₂;
X is O or NH;
R² stands for a linear or branched C₁₋₃₀-alkyl radical;
R³ stands for a linear or branched C₁₋₂₀-alkyl radical;
all R⁴, independently of one another, stand for H or linear or branched C₁₋₈alkyl radicals;
R⁵ stands for H, a linear or branched C₁₋₈-alkyl radical or a linear or branched —O—C₁₋₈-alkyl radical; and
R⁶ stands for a C₁₋₈-alkyl radical,
   where the photostabiliser is particularly preferably bis(2-ethylhexyl)2-(4-hydroxy-3,5-dimethoxybenzylidene)malonate. Corresponding photostabilisers and their preparation and use are described in International patent application WO 03/007906, the disclosure content of which expressly also belongs to the subject-matter of the present application.

The compositions according to the invention can be prepared by processes which are well known to the person skilled in the art, in particular by the processes which serve for the preparation of oil-in-water emulsions or water-in-oil emulsions.

The present invention furthermore relates to compositions having light-protection properties comprising the powder-form UV filters according to the invention and one or more cosmetically or dermatologically suitable carriers, a process for the preparation of a composition which is characterised in that at least one powder according to the invention is mixed with a cosmetically or dermatologically suitable carrier, and the use of powder-form UV filters according to the invention for the preparation of a composition having light-protection properties.

These compositions can be, in particular, in the form of simple or complex emulsions (O/W, W/O, O/W/O or W/O/W), such as creams, milks, gels or gel creams, powders and solid sticks, and they may, if desired, be formulated as aerosols and be in the form of foams or sprays. These compositions are preferably in the form of an O/W emulsion.

The cosmetic compositions according to the invention can be used as compositions for protection of the human epidermis or of the hair against UV radiation, as sunscreen compositions or make-up products.

It should be pointed out that in the formulations according to the invention for sun protection which have a carrier of the oil-in-water emulsion type, the aqueous phase (which comprises, in particular, the hydrophilic filters) generally makes up 50 to 95% by weight and preferably 70 to 90% by weight, based on the formulation as a whole, the oil phase (which comprises, in particular, the lipophilic filters) makes up 5 to 50% by weight and preferably 10 to 30% by weight, based on the formulation as a whole, and the (co)emulsifier or (co)emulsifiers make(s) up 0.5 to 20% by weight and preferably 2 to 10% by weight, based on the formulation as a whole.

Suitable compositions are those for external use, for example in the form of a cream, lotion or gel or as a solution which can be sprayed onto the skin. Suitable for internal use are administration forms such as capsules, coated tablets, powders, tablet solutions or solutions.

Examples which may be mentioned of application forms of the compositions according to the invention are: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower products. Any desired customary carriers, auxiliaries and, if desired, further active ingredients may be added to the composition.

Preferred auxiliaries originate from the group of the preservatives, antioxidants, stabilisers, solubilisers, vitamins, colorants and odour improvers.

Ointments, pastes, creams and gels may comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary carriers, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, corn oil, olive oil, castor oil and sesame oil, glycerol fatty acid esters, polyethylene glycols and fatty acid esters of sorbitan, or mixtures of these substances.

Suspensions may comprise the customary carriers, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary carriers, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary carriers, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary carriers, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, mascara, eyeliner, eye shadow, rouge, powder make-up, emulsion make-up and wax make-up, and sunscreen, pre-sun and after-sun preparations. The preferred composition forms according to the invention include, in particular, emulsions.

Emulsions according to the invention are advantageous and comprise, for example, the said fats, oils, waxes and other fatty bodies, as well as water and an emulsifier, as usually used for a composition of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids;
silicone oils, such as, for example, dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and waxes, silicone oils, dialkyl ethers, or the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low C number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty bodies, as well as water and an emulsifier, as usually used for a formulation of this type.

In a preferred embodiment, the compositions according to the invention comprise hydrophilic surfactants.

The hydrophilic surfactants are preferably selected from the group of the alkylglucosides, acyl lactylates, betaines and coconut amphoacetates.

The alkylglucosides are themselves advantageously selected from the group of the alkylglucosides which are distinguished by the structural formula 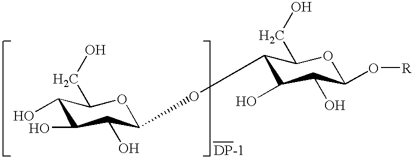
where R represents a branched or unbranched alkyl radical having 4 to 24 carbon atoms and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ to pᵢ represent the proportions of mono-, di-, tri- to i-fold glucosylated products in percent by weight. Products having degrees of glucosylation of 1-2, particularly advantageously of 1.1 to 1.5, very particularly advantageously of 1.2-1.4, in particular of 1.3, are advantageously selected in accordance with the invention.

The value D̅P̅ takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglucosides which are particularly advantageously used in accordance with the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and auxiliaries or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group of the substances which are distinguished by the structural formula 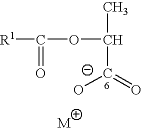
where R¹ denotes a branched or unbranched alkyl radical having 1 to 30 carbon atoms and M⁺ is selected from the group of the alkali metal ions and the group of the ammonium ions which are substituted by one or more alkyl and/or by one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group of the substances which are distinguished by the structural formula 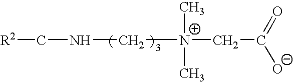
where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageously selected in accordance with the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied to the skin and/or the hair in an adequate amount in the usual manner for cosmetics.

Cosmetic and dermatological compositions according to the invention may exist in various forms. Thus, they can be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

An emulsifier that has proven to be particularly preferred in accordance with the invention for O/W emulsions is the commercial product Ceralution C from Sasol.

Co-emulsifiers which are advantageously selected in accordance with the invention are, for example, O/W emulsifiers, principally from the group of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or if isoalkyl derivatives are present, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention: fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Compositions which are preferred in accordance with the invention are particularly suitable for protecting human skin against UV-induced ageing processes and against oxidative stress, i.e. against damage caused by free radicals, as are generated, for example, by sunlight, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily/alcoholic, oily/aqueous or aqueous/alcoholic gels or solutions, in the form of solid sticks or may be formulated as an aerosol.

The composition may comprise cosmetic adjuvants that are usually used in this type of composition, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty body, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which, apart from the compound(s) of the formula I, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily/alcoholic gels also comprise natural or synthetic oil or wax.

The solid sticks consist of natural or synthetic waxes and oils, fatty alcohols, fatty acids, fatty acid esters, lanolin and other fatty bodies.

If a composition is formulated as an aerosol, the customary propellants, such as alkanes, fluoroalkanes and chlorofluoroalkanes, are generally used.

The cosmetic composition may also be used to protect the hair against photochemical damage in order to prevent colour changes, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling and treating the hair, in the form of a lotion or gel for brushing or laying a water wave, in the form of a hair lacquer, permanent-waving composition, colorant or bleach for the hair. The composition having light-protection properties may comprise various adjuvants used in this type of composition, such as surface-active agents, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The following examples are intended to explain the invention in greater detail, but without restricting it.

### EXAMPLES

### Example 1

### Spray-Drying with Two-Component Nozzle

The spray dryer with two-component nozzle (model Mobile Miono 2000 D from Niro) is heated to an entry temperature of 150° C. and an exit temperature of 75° C. In order to stabilise the system, water is sprayed in over a period of 10 minutes, then 5 kg of a dispersion of Eusolex® UV-Pearls OMC (Merck KGaA) in water (solids content 40% by weight) are atomised. After 30 minutes, the first runnings of product are removed, and after 3.5 hours the entire dispersion has been atomised. Spraying is continued with water for about 15 minutes. 1640 g of spray material are obtained.

Process Parameters:

Two-component nozzle (quantity of atomisation air 5 kg/h (0.3 bar))
Countercurrent mode
Product discharge: two-point deposition (dryer cone & cyclone)
Quantity of drying air: 85 kg/h
Exhaust temperature: 50° C.
Feed quantity of dispersion: 1.2 kg/h (conveyance by means of hose pump)

### Example 2

### Variant with Atomiser Wheel

The spray dryer with atomiser wheel (operating pressure 4.6 bar, 25000 rpm) is heated to an entry temperature of 150° C. and an exit temperature of 70° C. For stabilisation of the systems, water is sprayed in over a period of 10 minutes, then 5 kg of a dispersion of Eusolex® UV-Pearls OMC (Merck KGaA) in water (solids content 40% by weight) is atomised. After 30 minutes, the first runnings of product are removed, and after 3.5 hours the entire dispersion has been atomised. Spraying is continued with water for about 15 minutes. 1960 g of spray material are obtained.

Process Parameters:

Atomiser wheel
Co-current mode
Product discharge: two-point deposition (dryer cone & cyclone)
Quantity of drying air: 85 kg/h
Exhaust temperature: 50° C.
Feed quantity of dispersion: 1.2 kg/h (conveyance by means of hose pump)

Example Recipe of Face Powder:

| | | |
|---|---|---|
| Raw material | INCI | [%] |

A

| | | |
|---|---|---|
| Product from Example 1 or 2 | ETHYLHEXYL | 5.00 |
| | METHOXYCINNAMATE, | |
| | SILICA, PVP, CHLORPHENESIN | |
| | BHT, AQUA (WATER) | |
| Microna ® Matte Yellow | MICA, CI 77492 (IRON OXIDES) | 2.60 |
| Microna ® Matte Red | MICA, CI 77491 (IRON OXIDES) | 0.70 |
| Microna ® Matte Orange | MICA, CI 77491 (IRON OXIDES) | 0.80 |
| Microna ® Matte Black | MICA, CI 77499 (IRON OXIDES) | 0.30 |
| Magnesium stearate | MAGNESIUM STEARATE | 2.00 |
| Satin mica | MICA | 15.00 |
| Talc | TALC | 69.60 |

B

| | | |
|---|---|---|
| Ceraphyl 368 | ETHYLHEXYL PALMITATE | 3.92 |
| Propyl 4-hydroxybenzoate | PROPYLPARABEN | 0.08 |

Preparation:

The constituents of the powder base are introduced into the mixer (La Moulinette from Moulinex) and mixed for 2×10 seconds. The mixture is transferred into a beaker, the binder is added dropwise, and the mixture is stirred using a spatula. The mixture is again introduced into the mixer and mixed for 3×10 seconds. The powder is pressed at 20 bar.

Comparative Example of Face Powder:

| | | |
|---|---|---|
| Raw material | INCI | [%] |

A

| | | |
|---|---|---|
| Microna ® Matte Yellow | MICA, CI 77492 (IRON OXIDES) | 2.60 |
| Microna ® Matte Red | MICA, CI 77491 (IRON OXIDES) | 0.70 |
| Microna ® Matte Orange | MICA, CI 77491 (IRON OXIDES) | 0.80 |
| Microna ® Matte Black | MICA, CI 77499 (IRON OXIDES) | 0.30 |
| Magnesium stearate | MAGNESIUM STEARATE | 2.00 |
| Satin mica | MICA | 15.00 |
| Talc | TALC | 74.60 |

B

| | | |
|---|---|---|
| Eusolex ® 2292 | ETHYLHEXYL | 2.40 |
| | METHOXYCINNAMATE, BHT | |
| Ceraphyl 368 | ETHYLHXYL PALMITATE | 1.52 |
| Propyl 4-hydroxybenzoate | PROPYLPARABEN | 0.08 |

Preparation:

The constituents of the powder base are introduced into the mixer (La Moulinette from Moulinex) and mixed for 2×10 seconds. The mixture is transferred into a beaker, the binder is added dropwise, and the mixture is stirred using a spatula. The mixture is again introduced into the mixer and mixed for 3×10 seconds. The powder is pressed at 20 bar.

On use of the powder according to the invention, freer formulation ability arises compared with that from the comparative example, in which some of the binder has to be replaced by the liquid UV filter. This can result in restriction during formulation, for example with respect to the skin feel.

Example Recipe of Hydrogel:

Day care for greasy skin
Oil-free, SPF (DIFFEY) 7, UVA-PF 3

| | | |
|---|---|---|
| Raw material | INCI | [%] |

A

| | | |
|---|---|---|
| Product from Example | ETHYLHEXYL | 8.00 |
| 1 or 2 | METHOXYCINNAMATE, SILICA, | |
| | PVP, CHLORPHENESIN, BHT, AQUA | |
| | (WATER), | |
| Hispagel 200 | GLYCERIN, GLYCERYL | 25.00 |
| | POLYACRYLATE | |
| RonaCare ® ectoine | ECTOIN | 0.50 |
| Water, demineralised | AQUA (WATER) | 65.80 |
| Germaben II | PROPYLENE GLYCOL, | 0.70 |
| | DIAZOLIDINYL UREA, | |
| | METHYLPARABEN, | |
| | PROPYLPARABEN | |

Preparation:

Initially introduce Hispagel. Dissolve ectoine in water, add remaining constituents and add to the Hispagel with stirring.

Stir until a homogeneous mixture has formed.

Notes:

pH (25° °C.)=5.5
Viscosity (Brookfield RVD II, Helipath spindle C, 10 rpm, 25° C.)=45,900 cps

Example Recipe of O/W (Incorporation into the Water Phase):

O/W sunscreen lotion
SPF (in vitro, Diffey method) 22 ± 4, UVA-PF 13 ± 3

| | | |
|---|---|---|
| Raw material | INCI | [%] |

A

| | | |
|---|---|---|
| Eusolex ® 6300 | 4-METHYLBENZYLIDENE | 4.00 |
| | CAMPHOR | |
| Eusolex ® 9020 | BUTYL | 5.00 |
| | METHOXYDIBENZOYLMETHANE | |
| Hostacerin DGI | POLYGLYCERYL-2 | 4.00 |
| | SESQUIISOSTEARATE | |
| Cetiol B | DIBUTYL ADIPATE | 4.00 |
| Crodamol DOA | DIOCTYL ADIPATE | 2.00 |
| Tegosoft TN | C12-15 ALKYL BENZOATE | 4.00 |
| Eutanol G | OCTYLDODECANOL | 4.50 |

B

| | | |
|---|---|---|
| Aristoflex AVC | AMMONIUM | 0.50 |
| | ACRYLOYLDIMETYLTAURATE/ | |
| | VP COPOLYMER | |

C

| | | |
|---|---|---|
| Water, demineralised | AQUA (WATER) | 40.00 |

D

| | | |
|---|---|---|
| Product from Example | ETHYLHEXYL | 8.40 |
| 1 or 2 | METHOXYCINNAMATE, SILICA, | |
| | PVP, CHLORPHENESIN, | |
| | BHT AQUA (WATER), | |
| Hostapon KCG | SODIUM COCOYL GLUTAMATE | 1.50 |
| Panthenol-D | PANTHENOL | 0.50 |
| Glycerol, anhydrous | GLYCERIN | 3.00 |
| Water, demineralised | AQUA (WATER) | 17.40 |

E

| | | |
|---|---|---|
| Phenonip | PHENOXYETHANOL, | 0.70 |
| | BUTYLPARABEN, | |
| | ETHYLPARABEN, PROPYLPARABEN, | |
| | METHYLPARABEN | |
| RonaCare ® | TOCOPHERYL ACETATE | 0.50 |
| tocopherol acetate | | |
| Perfume oil (q.s.) | PARFUM | 0.00 |

Preparation:
Heat A to 80° C.
Stir B into A
Warm C to 80° C.
Prepare intermediate W/O emulsion:
Stir C into A/B at high shear rate (Ultra Turrax) for about 2 minutes
Add cold phase D dropwise very slowly until phase conversion takes place, stir cold for at least 2 h
Add E and stir for a further hour
Homogenise.
   Notes:
pH (25° C.)=6.0
Viscosity (Brookfield RVD II, Helipath spindle B, 50 rpm, 25° C.)=1.120 cps

Example Recipe of W/O (Incorporation into the Oil Phase):

Complete sun protection (W/O)
in vitro SPF (Diffey, Transpare, SPF290) = 40 +/− 9, lambda crit.
(DGK) = 373 nm

| | | |
|---|---|---|
| Raw material | INCI | [%] |

A

| | | |
|---|---|---|
| Product from | ETHYLHEXYL METHOXYCINNAMATE | 4.00 |
| Example 1 or 2 | SILICA, PVP, CHLORPHENESIN, BHT | |
| | AQUA (WATER), | |
| Eusolex ® 6300 | 4-METHYLBENZYLIDENE CAMPHOR | 1.00 |
| Eusolex ® 9020 | BUTYL METHOXYDIBENZOYLMETHANE | 1.00 |
| Cremophor | PEG-7 HYDROGENATED CASTOR OIL | 6.00 |
| WO 7 | | |
| Elfacos ST 9 | PEG-45 DODECYL GLYCOL COPOLYMER | 2.00 |
| Jojoba oil | BUXUS QUINENSIS (JOJOBA OIL) | 9.00 |
| Isopropyl | ISOPROPYL MYRISTATE | 3.50 |
| myristate | | |
| Abil 350 | DIMETHICONE | 1.00 |

B

| | | |
|---|---|---|
| Eusolex ® | TITANIUM DIOXIDE, SILICA | 5.00 |
| T-AVO | | |

C

| | | |
|---|---|---|
| Glycerol (87% | GLYCERIN | 5.00 |
| extra pure) | | |
| Titriplex ® III | DISODIUM EDTA | 0.20 |
| Water, | AQUA (WATER) | 61.50 |
| demineralised | | |
| Germall 115 | IMIDAZOLIDINYL UREA | 0.30 |

D

| | | |
|---|---|---|
| Phenonip | PHENOXYETHANOL, BUTYLPARABEN, | 0.50 |
| | ETHYLPARABEN, PROPYLPARABEN, | |
| | METHYLPARABEN | |
| Perfume oil (q.s.) | PARFUM | 0.00 |

Preparation:
Heat phase A to 80°° C., introduce phase B with stirring and homogenise for 3 minutes (Zauberstab setting 1).

Heat phase C to 80° C. and homogenise into phase A/B (1 minute Zauberstab setting 1 & 30 s setting 2.

Cool to 40° C. with stirring, add phase D and homoginise again (MFR stirrer 1200 rpm 1 minute).

Notes:

Viscosity (Brookfield RVT-DV II, Helipath C. 10 rpm, 24° C.)=11,700 mPa s

Example Recipe of O/W (Incorporation into the Oil Phase):

Sunscreen lotion, water-resistant (O/W)
expected SPF approx. 25

| | | |
|---|---|---|
| Raw material | INCI | [%] |

A

| | | |
|---|---|---|
| Product from | ETHYLHEXYL | 6.00 |
| Example 1 or 2 | METHOXYCINNAMATE, BHT | |
| | SILICA, PVP, | |
| | CHLORPHENESIN, BHT | |
| | AQUA (WATER), | |
| Eusolex ® 6300 | 4-METHYLBENZYLIDENE CAMPHOR | 1.00 |
| Eusolex ® 9020 | BUTYL | 3.00 |
| | METHOXYDIBENZOYLMETHANE | |
| Amphisol A | CETYL PHOSPHATE | 2.00 |
| Lanette O | CETEARYL ALCOHOL | 0.50 |
| Cutina GMS | GLYCERYL STEARATE | 4.00 |
| Dow Corning 200 | DIMETHICONE | 0.50 |
| (100 cs) | | |
| Crodamol AB | C12-15 ALKYL BENZOATE | 9.00 |
| Antaron WP-660 | TRICONTANYL PVP | 3.00 |

B

| | | |
|---|---|---|
| Eusolex ® 232 | PHENYLBENZIMIDAZOLE | 2.00 |
| | SULFONIC ACID | |
| Tris(hydroxymethyl)- | TROMETHAMINE | 0.90 |
| aminomethane | | |
| Water, demineralised | AQUA (WATER) | 62.20 |
| Carbopol 980, | AQUA, CARBOMER | 5.00 |
| 2% solution, | | |
| neutralised to pH 7 | | |

C

| | | |
|---|---|---|
| Perfume (q.s.) | PARFUM | 0.20 |
| Germaben II | PROPYLENE GLYCOL, | 0.70 |
| | DIAZOLIDINYL UREA, | |
| | METHYLPARABEN, | |
| | PROPYLPARABEN | |
| Sodium hydroxide, | SODIUM HYDROXIDE | 0.00 |
| 10% solution | | |

Preparation:

In order to neutralise Eusolex® 232, tris(hydroxymethyl)aminomethane is dissolved in the water of phase B, and Eusolex® 232 is added with stirring. Add the remaining constituents of phase B uniformly and heat to 80° C. Phase A is heated to 75° C. Phase B is slowly added to phase A with gentle stirring and hemogenised. Cool to 40° C. with stirring, add phase C and adjust pH to 7.

Notes:

pH (25° C.)=6.9

Viscosity (Brookfield RVT-DV II, Helipath C, 10 rpm, 25° C.)=55,000 cps

Example Recipe of W/O (Incorporation into the Water Phase):

Sunscreen spray lotion (W/O)

| | | |
|---|---|---|
| Raw material | INCI | [%] |

A

| | | |
|---|---|---|
| Arlacel P135 | PEG-30 DIPOLYHYDROXYSTEARATE | 3.00 |
| Cetiol A | HEXYL LAURATE | 5.50 |
| Arlamol HD | ISOHEXADECANE | 8.00 |
| Miglyol 812 N | CAPRYLIC/CAPRIC TRIGLYCERIDE | 4.00 |
| Arlamol DOA | DIOCTYL ADIPATE | 4.00 |

B

| | | |
|---|---|---|
| Product from | ETHYLHEXYL | 8.00 |
| Example 1 or 2 | METHOXYCINNAMATE, | |
| | SILICA, PVP, | |
| | CHLORPHENESIN, BHT | |
| | AQUA (WATER), | |
| RonaCare ® ectoine | ECTOIN | 0.10 |
| Magnesium sulfate | MAGNESIUM SULFATE | 0.70 |
| heptahydrate | | |
| Glycerol (about 87%) | GLYCERIN | 3.00 |
| Titriplex ® III | DISODIUM EDTA | 0.05 |
| Water, demineralised | AQUA (WATER) | 57.65 |

C

| | | |
|---|---|---|
| Eusolex ® T-S | TITANIUM DIOXIDE, ALUMINA, | 5.00 |
| | STEARIC ACID | |

D

| | | |
|---|---|---|
| Phenonip | PHENOXYETHANOL, | 0.70 |
| | BUTYLPARABEN, ETHYLPARABEN, | |
| | PROPYLPARABEN, | |
| | METHYLPARABEN | |
| Fragrance | PARFUM | 0.30 |
| L'EAU D'ETE + | | |
| D12921CT | | |

Preparation:

Phases A and B are combined separately and heated to 80° C. Phase B is slowly added to phase A with vigorous stirring and homogenised. Cool to 40° C. with stirring and add phase C. After homogeneous dispersion of Eusolex® T-S, add phase D and cool with stirring.

Notes:

Viscosity 5,000 mPas (Brookfield LV, spindle 4; 12 rpm).

Sunscreen Care Lotion:

| | | |
|---|---|---|
| Raw material | INCI | [%] |

A

| | | |
|---|---|---|
| Liquid paraffin | PARAFFINUM LIQUIDUM | 6.50 |
| Pelemol BIP | ISOPROPYLPHTALIMIDE, | 6.00 |
| | BUTYLPHTALIDE | |
| Isopropyl palmitate | ISOPROPYL PALMITATE | 7.50 |
| Soya oil | GLYCINE SOJA | 5.00 |
| RonaCare ® | TOCOPHEROL ACETAT | 1.00 |
| tocopherol acetate | | |
| Carbopol Ultrez 10 | CARBOMER | 0.30 |

B

| | | |
|---|---|---|
| Product from Example 1 | ETHYLHEXYL | 5.60 |
| or 2 | METHOXYCINNAMATE, | |
| | SILICA, PVP, | |
| | CHLORPHENESIN, BHT | |
| | AQUA (WATER) | |
| Product from | HOMOSALATE, | 5.60 |
| Example 1 or 2 | BUTYLMETHOXY | |
| | DIBENZOYL-METHANE, | |
| | DIETHYLHEXYL | |
| | SYRINGYLIDENE MALONATE | |
| | SILICA, PVP, CHLORPHENESIN, | |
| | BHT AQUA | |
| Water, demineralised | AQUA (WATER) | 54.20 |
| Sisterna L70-C | SUCROSE LAURATE, AQUA, | 6.00 |
| | ALCOHOL | |
| Phenonip | PHENOXYETHANOL, | 1.00 |
| | BUTYLPARABENE, | |
| | METHYLPARABENE, | |
| | PROPYLPARABENE, | |
| | ETHYLPARABENE | |

C

| | | |
|---|---|---|
| Sodium hydroxide | SODIUM HYDROXIDE, AQUA | 1.20 |
| solution, 10% | (WATER) | |

D

| | | |
|---|---|---|
| Perfume oil (q.s.) | PARFUME, FRAGANCE | 0.30 |
| | | 100.00 |

Preparation:

Combine phase A apart from the Carbopol. If necessary, warm to about 50° C. Incorporate the Carbopol and emulsify pre-dissolved phase B in with stirring. Homogenise. After addition of phase C, briefly homogenise again and add phase D.

1. UV filters in powder form, obtainable by spray-drying or freeze-drying a dispersion comprising UV filters.

2. UV filters in powder form according to claim 1, characterised in that the dispersion is aqueous.

3. UV filters in powder form according to claim 1, characterised in that the UV filters are encapsulated and/or unencapsulated organic UV filters.

4. UV filters in powder form according to claim 1, characterised in that the UV filters are encapsulated organic UV filters.

5. UV filters in powder form according to claim 4, characterised in that the walls of the capsules are inorganic.

6. UV filters in powder form according to claim 5, characterised in that the walls of the capsules are built up from silica gel or silicon dioxide.

7. UV filters according to claim 4, characterised in that the capsules comprise further substances.

8. UV filters according to claim 7, characterised in that the substances are photostabilisers, cosmetic oils and/or antioxidants.

9. UV filters in powder form according to claim 1, characterised in that the powder is additionally after-treated.

10. Process for the preparation of UV filters in powder form according to claim 1, characterised in that dispersions of UV filters are spray-dried or freeze-dried.

11. Process according to claim 10, characterised in that the UV filters are encapsulated and/or unencapsulated organic UV filters.

12. Process according to claim 10, characterised in that the UV filters are encapsulated organic UV filters.

13. Process according to claim 10, characterised in that the UV filters are in particulate form.

14. Process according to claim 13, characterised in that the size of the UV filters in particulate form is 10 nm to 100 μm.

15. Process according to claim 10, characterised in that additives are introduced before or during the process.

16. Process according to claim 10, characterised in that the UV filters in powder form are after-treated.

17. Compositions having light-protection properties comprising UV filters in powder form according to claim 1.

18. Composition according to claim 17, characterised in that it is a composition which can be applied topically.

19. Composition according to claim 17, characterised in that it is a composition for decorative cosmetics.

20. Composition having light-protection properties according to claim 1, characterised in that the composition additionally comprises at least one organic and/or inorganic UV filter.

21. Composition having light-protection properties according to claim 1, characterised in that the composition comprises at least one self-tanning agent.

22. Composition having light-protection properties according to claim 1, characterised in that the composition comprises at least one photostabiliser.

23. Composition according to claim 22, characterised in that the photostabiliser is selected from the compounds conforming to the formula V 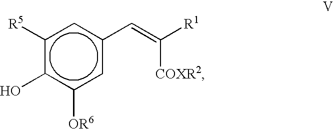
where
R¹ is selected from —C(O)CH₃, —CO₂R³, —C(O)NH₂ and —C(O)N(R⁴)₂;
X is O or NH;
R² stands for a linear or branched C₁₋₃₀-alkyl radical;
R³ stands for a linear or branched C₁₋₂₀-alkyl radical,
all R⁴, independently of one another, stand for H or linear or branched C₁₋₈-alkyl radicals
R⁵ stands for H, a linear or branched C₁₋₈-alkyl radical or a linear or branched —O—C₁₋₈-alkyl radical and
R⁶ stands for a C₁₋₈-alkyl radical.

24. Corn position having light-protection properties according to claim 1, characterised in that the composition comprises one or more further UV filters selected from the group consisting of 3-(4′-methylbenzylidene)-dl-camphor, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid, and the potassium, sodium and triethanolamine salts thereof.

25. Composition having light-protection properties according to claim 1, suitable for the protection of body cells against oxidative stress, characterised in that it preferably comprises one or more antioxidants.

26. Composition having light-protection properties according to claim 1, characterised in that it is an emulsifier-free emulsion.

27. Process for the preparation of a corn position, characterised in that a UV filter according to claim 1 is mixed with a cosmetically or dermatologically suitable carrier and optionally further ingredients.
